(19) United States
(12) Patent Application Publication
(10) Pub. No.: US 2023/0127442 A1
(43) Pub. Date: Apr. 27, 2023

(54) COMPOSITIONS FOR INCREASING ETHANOL PRODUCTION AND RELATED METHODS

(71) Applicant: KEMIN INDUSTRIES, INC., DES MOINES, IA (US)

(72) Inventor: Brandon LEWIS, Johnston, IA (US)

(73) Assignee: KEMIN INDUSTRIES, INC., DES MOINES, IA (US)

(21) Appl. No.: 17/974,506

(22) Filed: Oct. 26, 2022

Related U.S. Application Data
(60) Provisional application No. 63/344,170, filed on May 20, 2022.
(60) Provisional application No. 63/272,014, filed on Oct. 26, 2021.

(51) Int. Cl. C12P 7/06

(52) U.S. Cl. CPC C12P 7/06

(57) ABSTRACT

The present invention relates to methods and compositions capable of counteracting heat and ethanol stress to yeast during an ethanol production process. Another aspect of the present invention relates to methods for increasing ethanol production yield by at least 0.5%. Other aspects of the present invention relate to supporting yeast during a fermentation process and reducing overall input costs for ethanol production.

### CROSS-REFERENCE TO RELATED APPLICATIONS

The present application claims the benefit of priority to U.S. Provisional Patent Application No. 63/344,170, filed May 20, 2022, entitled “COMPOSITIONS FOR INCREASING ETHANOL PRODUCTION AND RELATED METHODS,” and U.S. Provisional Patent Application No. 63/272,014, filed Oct. 26, 2021, entitled “COMPOSITIONS FOR INCREASING ETHANOL PRODUCTION AND RELATED METHODS,” the entire disclosures of which are hereby incorporated by reference in their entireties.

### BACKGROUND OF THE INVENTION

According to the U.S. Department of Energy, the United States leads the world in ethanol production, generating nearly 14 billion gallons of ethanol in 2020. Given the importance of this industry, there has been significant focus on improving the efficiency of the production of ethanol from corn or grain. This includes optimizing the manufacturing process, as well as reducing the costs associated with the production of ethanol.

The production process of ethanol is well known. Jacques, K., Lyons, T., and Kelsall, D. 2003, The Alcohol Textbook, 4th Edition, Nottingham University Press, United Kingdom. Ethanol production includes a fermentation step that requires yeast. During fermentation, the yeast is exposed to physical and chemical stressors that lead to reduced cell growth or death and changes in yeast metabolism. These physical and chemical stressors include heat and ethanol, which are both generated by yeast during fermentation. These stressors are generally understood to impact and limit yield.

For instance, during industrial fermentations, yeast generate excessive heat. In the absence of cooling, the yeast fermentation efficiency is significantly affected. For that reason, ethanol production facilities have typically relied on cooling towers to reduce the negative consequences of metabolic fermentation heat generation; however, during the summer months the cooling towers may be overwhelmed. Birch, R. M., and Walker, G. M., Influence of magnesium ions on heat shock and ethanol stress responses of Saccharomyces cerevisiae, Enzyme Microb. Technol. 26, 678-687 (2000). When this occurs, the facility may take several actions to seek to counteract the problem, including lowering the sugar levels and corn solids going to fermentation, adjusting nitrogen availability, and using more robust yeast. Unfortunately, these actions translate into increased operating costs for the facility and potentially reduce ethanol yield. Geddes, C., Running Hot: 7 tips for warm weather ethanol fermentation, Biofuels Digest (2017).

Additionally, yeast is also endangered in the presence of high levels of ethanol, sometimes referred to as “ethanol stress.” As ethanol is produced by the yeast, it accumulates in the growth medium inhibiting yeast growth and cell division. As ethanol levels continue to increase cell death occurs. Stanley, D., Bandara, A., Fraser, S., Chambers, P. J., and Stanley, G. A., The ethanol stress response and ethanol tolerance of Saccharomyces cerevisiae, J. Appl. Microbiol. 109, 13-24 (2010). It follows, managing cell division and growth is critical. Researchers have only recently started to appreciate that late-stage ethanol production is not necessarily due to increased production efficiency; rather it relates to the amount of yeast at the end of fermentation. Interestingly, many of the adjustments facilities have taken to counter heat stress are applicable to countering ethanol stress but come with the same drawback of increased operating costs. Earls, D., How EtOH production affects yeast health: 4 tips for dealing with ethanol stress, Biofuels Digest (2017).

When yeast is exposed to excess heat (heat stress) or ethanol several metabolic changes occur. These include the production of heat shock proteins (Hsps), glutathione, superoxide dismutase, accumulation of intercellular trehalose and glycerol, modification of membrane lipid composition and ion exchange processes, and changes to the plasma membrane ATPase activity. Birch, R. M., and Walker, G. M., Influence of magnesium ions on heat shock and ethanol stress responses of Saccharomyces cerevisiae, Enzyme Microb Technol 26, 678-687 (2000). Hsps are now known to be induced when cells are exposed to other stressors than heat including ethanol. Hsps production is associated with increased tolerance to heat and ethanol. It has been observed that when yeast is pre-exposed to moderate heat, the yeast was able to withstand longer exposure to higher levels of heat or ethanol likely due to the induction of Hsps during the moderate exposure. Sanchez, Y., Taulien, J., Borkovich, K. A., and Lindquist, S., Hsp104 is required for tolerance to many forms of stress, EMBO J 11, 2357-2364 (1992).

While inherent glutathione and superoxide dismutase are involved in the reduction of oxidative damage to the cell, there is an absence of studies or research considering the efficacy of supplemental antioxidants and their potential to increase ethanol production by yeast under heat stress or ethanol stress. The present invention discloses for the first time that supplementation of antioxidants, specifically those derived from plant sources, and synthetic antioxidants are capable of supporting yeast and can significantly increase overall ethanol production.

### SUMMARY OF THE INVENTION

The present invention relates to compositions capable of counteracting heat and ethanol stress to yeast during an ethanol production process or fermentation process. Another aspect of the present invention relates to methods for increasing ethanol production yield by at least 0.5%, or in some embodiments at least 1.5% or 2%. Another aspect of the present invention relates to supporting yeast during fermentation. Yet another aspect of the present invention relates to reducing overall input costs for industrial-scale ethanol production.

### DETAILED DESCRIPTION OF THE DRAWINGS

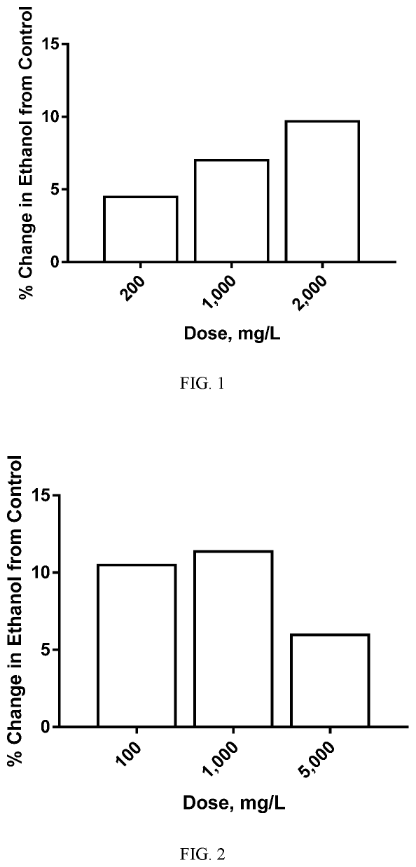

FIG. 1 depicts the change in ethanol production by yeast under heat stress supplemented with 200, 1,000, or 2,000 mg/L of lutein for 48 hours as a percentage of yeast with no lutein supplementation (control).

FIG. 2 depicts the change in ethanol production by yeast under heat stress supplemented with 100, 1,000, or 2,000 mg/L of aqueous tea extract for 48 hours as a percentage of yeast with no aqueous tea extract supplementation (control).

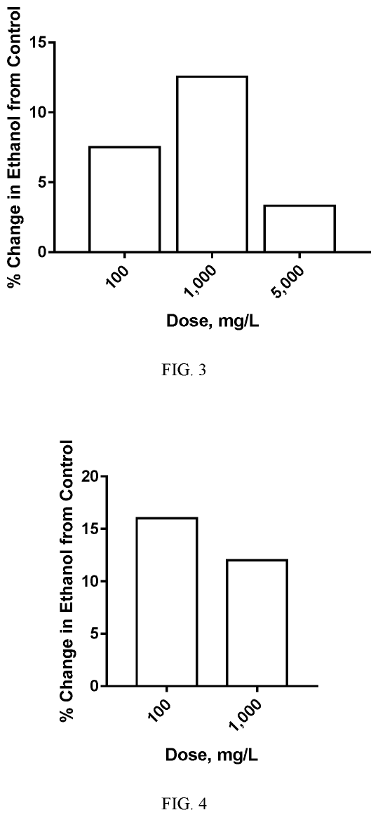

FIG. 3 depicts the change in ethanol production by yeast under heat stress supplemented with 100, 1,000, or 2,000 mg/L of spearmint extract for 48 hours as a percentage of yeast with no spearmint extract supplementation (control).

FIG. 4 depicts the change in ethanol production by yeast under heat stress supplemented with 100 or 1,000 mg/L of aqueous tea extract for 72 hours as a percentage of yeast with no aqueous tea extract supplementation (control).

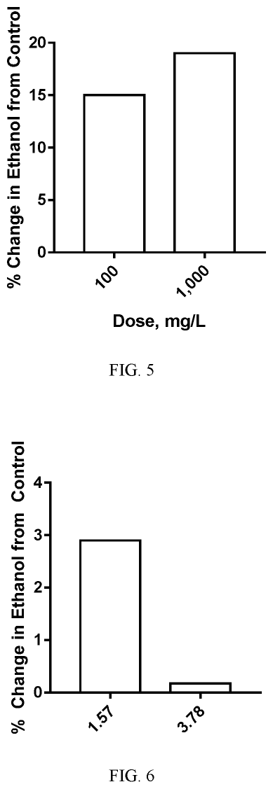

FIG. 5 depicts the change in ethanol production by yeast under heat stress supplemented with 100 or 1,000 mg/L of spearmint extract for 72 hours as a percentage of yeast with no spearmint extract supplementation (control).

FIG. 6 depicts the change in ethanol production by yeast under heat stress supplemented with 1.57 or 3.78 mg/L of spearmint extract for 72 hours as a percentage of yeast with no spearmint extract supplementation (control).

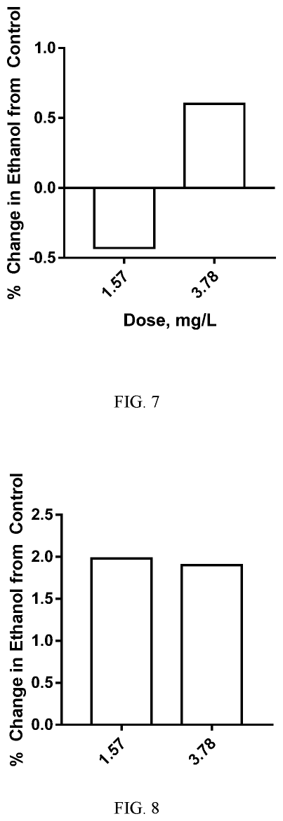

FIG. 7 depicts the change in ethanol production by yeast under heat stress supplemented with 1.57 or 3.78 mg/L of acerola extract for 72 hours as a percentage of yeast with no acerola extract supplementation (control).

FIG. 8 depicts the change in ethanol production by yeast under heat stress supplemented with 1.57 or 3.78 mg/L of solvent extracted tea for 72 hours as a percentage of yeast with no solvent extracted tea supplementation (control).

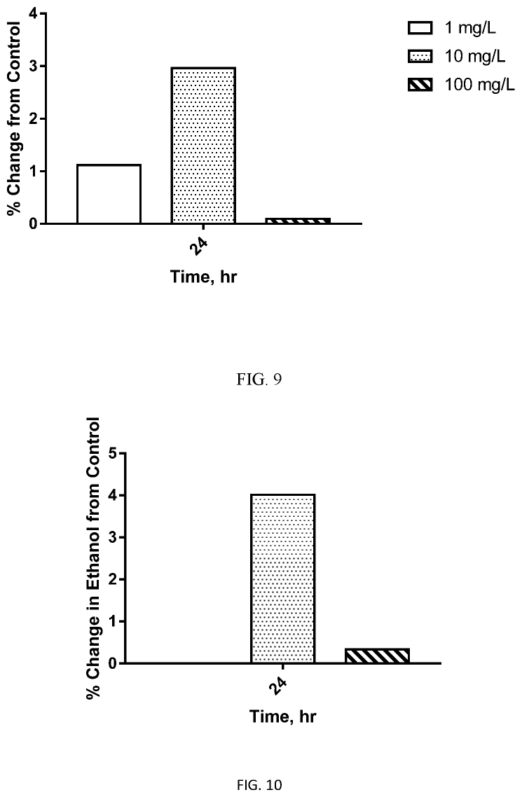

FIG. 9 depicts the change in ethanol production by yeast supplemented with 1, 10, or 100 mg/L of rosemary extract for 24 hours as a percentage of yeast with no rosemary extract supplementation (control).

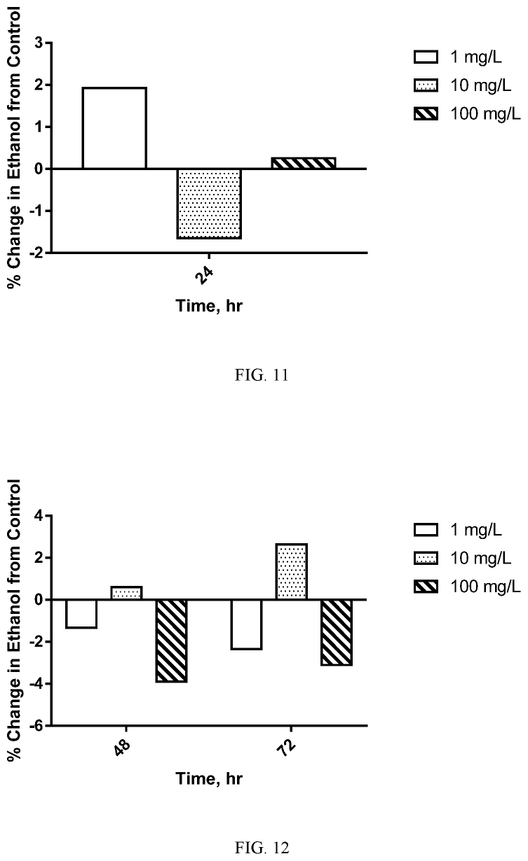

FIG. 10 depicts the change in ethanol production by yeast supplemented with 1, 10, or 100 mg/L of mixed tocopherols for 24 hours as a percentage of yeast with no mixed tocopherol supplementation (control).

FIG. 11 depicts the change in ethanol production by yeast supplemented with 1, 10, or 100 mg/L of lipid soluble tea extract for 24 hours as a percentage of yeast with no lipid soluble tea extract supplementation (control).

FIG. 12 depicts the change in ethanol production by yeast under heat stress supplemented with 1, 10, or 100 mg/L of rosemary extract for 72 hours as a percentage of yeast with no rosemary extract supplementation (control).

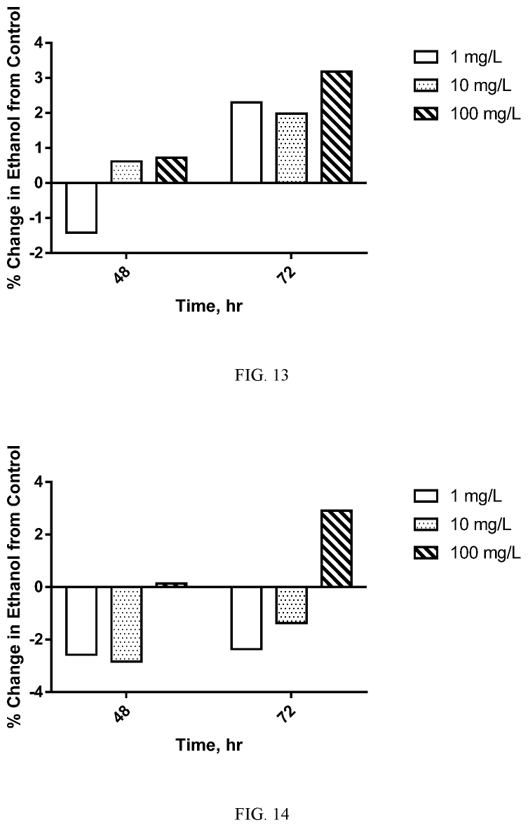

FIG. 13 depicts the change in ethanol production by yeast under heat stress supplemented with 1, 10, or 100 mg/L of mixed tocopherols for 72 hours as a percentage of yeast with no mixed tocopherols supplementation (control).

FIG. 14 depicts the change in ethanol production by yeast under heat stress supplemented with 1, 10, or 100 mg/L of lipid soluble tea extract for 72 hours as a percentage of yeast with no lipid soluble tea extract supplementation (control).

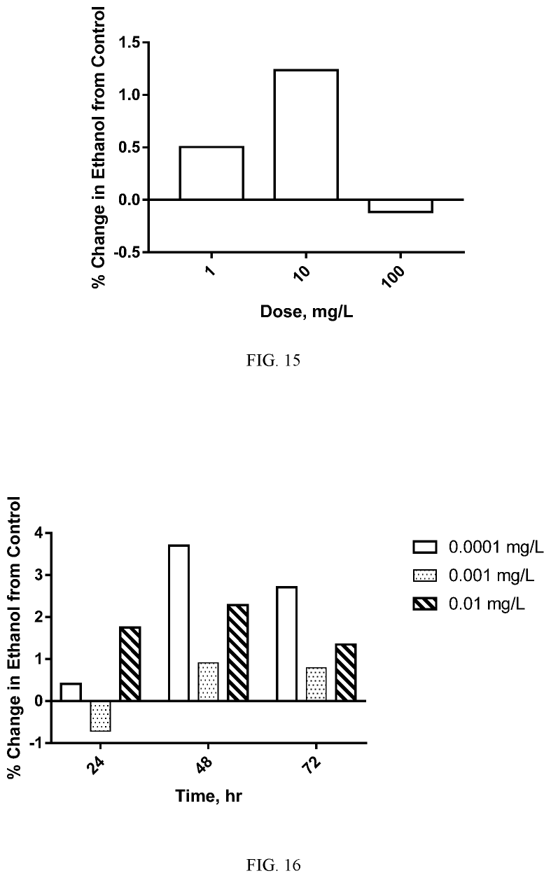

FIG. 15 depicts the change in ethanol production by yeast supplemented with 1, 10, or 100 mg/L solvent extracted tea for 72 hours as a percentage of yeast with no solvent extracted tea supplementation (control).

FIG. 16 depicts the change in ethanol production by yeast under heat stress supplemented with 0.0001, 0.001, or 0.01 mg/L of tert-butylhydroquinone for 72 hours as a percentage of yeast with no tert-butylhydroquinone supplementation (control).

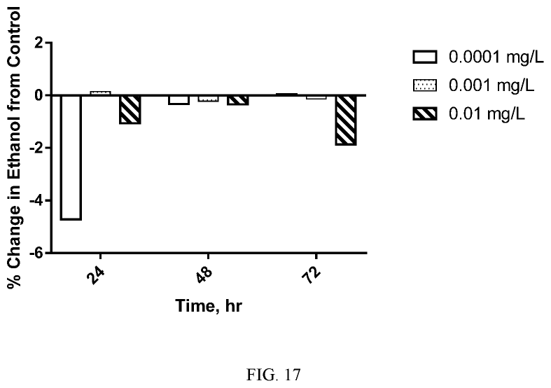

FIG. 17 depicts the change in ethanol production by yeast under heat stress supplemented with 0.0001, 0.001, or 0.01 mg/L of butylated hydroxytoluene for 72 hours as a percentage of yeast with no butylated hydroxytoluene supplementation (control).

### DETAILED DESCRIPTION OF THE INVENTION

The present invention relates to compositions capable of counteracting heat and/or ethanol stress in yeast during an ethanol production process, or a fermentation process. Another aspect of the present invention relates to methods for increasing ethanol production yield or reducing overall input costs for ethanol production.

As described in greater detail below, the inventor surprisingly found that the addition of a composition containing an effective amount of at least one antioxidant, or blends thereof, was beneficial to ethanol production yield. Additionally, the compositions and methods described were found to support yeast during a fermentation process by mitigating against heat or ethanol-induced stressors. Accordingly, additional studies were designed to determine whether the addition of antioxidants, including plant-based antioxidants and synthetic antioxidants, could improve ethanol production yield and protect yeast against metabolic stressors including heat and ethanol stress during industrial ethanol manufacturing processes.

In certain embodiments, the compositions or additives of the present invention contain at least one antioxidant in an amount effective to improve yield during ethanol production, for instance ethanol that can be used as a biofuel. In alternative embodiments, the antioxidant compositions of the present invention are used to improve yield during any fermentation step, including but not limited to the production of alcohol destined for human consumption. Additional studies were designed to confirm that the compositions of the present invention did not impact the flavor profile for certain applications, for instance in the production of alcoholic beverages, including but not limited to production of beer, wine, vodka, or whiskey.

In certain embodiments, the composition contains an antioxidant in an amount that supports the yeast during the fermentation step. In certain embodiments, the compositions of the present invention are added to the vessel where fermentation occurs, e.g., during the fermentation step. In alternative embodiments, the compositions of the present invention are added to the vessel where yeast propagation occurs. The compositions of the present invention can be added at the same time any other ingredient used during the fermentation is introduced.

In certain embodiments, the compositions contain at least one antioxidant that is a plant-based extract or derived from a plant source. For instance, lutein is a carotenoid extracted from marigolds, while acerola, tea, spearmint and rosemary extracts contain high levels of antioxidants and unique plant polyphenols. According to at least one embodiment, the lutein crystals are purified from marigolds and suspended in oil.

Spearmint extract has been shown to contain antioxidants, as well as polyphenols. According to at least one embodiment, the spearmint extract is an aqueous water extraction.

Acerola extract has been shown to contain high levels of antioxidants. According to at least one embodiment, the acerola extract is an aqueous water extraction.

Tea extract, for instance green tea extract, has also been shown to contain high levels of antioxidants. According to certain embodiments, the tea extract is extracted with water or organic solvent.

In alternative embodiments, the solvent includes water and modified water, including but not limited to acidified water, neutral pH water, basic pH water, and water with dissolved salts or water miscible organic solvents such as acetone, ethanol, glycerol, isopropanol, methanol, or combinations thereof.

In alternative embodiments, known organic solvents are used, including but not limited to organic solvents such as acetone, ethanol, ethyl acetate, hexane, isopropanol, or combinations thereof.

According to at least one embodiment, these plant-based antioxidant sources were used to demonstrate the efficacy of both water-soluble and fat-soluble antioxidants. The extracts contained a minimum amount of 20% by weight lutein in oil, a minimum 20% by weight catechins in the tea extracts, and a minimum of 24% by weight polyphenols in the spearmint extract. According to certain embodiments, the amount of lutein added to the fermentation step ranged from about 200 mg/L to 2,000 mg/L while the amount of tea and spearmint extracts ranged from about 100 mg/L to 5,000 mg/L. Alternative embodiments include antioxidants derived from other natural sources, including but not limited to antioxidants found in virtually all plants extracted using either water or organic solvents.

According to at least one embodiment, the composition of the present invention includes at least one synthetic antioxidant including but not limited to tert-butylhydroquinone (TBHQ), butylated hydroxytoluene (BHT), or blends thereof. In at least one embodiment, TBHQ is solubilized in propylene glycol. In at least one embodiment, BHT is solubilized in ethanol. In alternative embodiments, the at least one synthetic antioxidant could be suspended in liquid or solubilized in liquid, including but not limited to water and organic solvents or combinations thereof.

While it is preferred to add the compositions that contain at least one antioxidant during propagation or fermentation steps, antioxidants have been shown to maintain activity at temperatures higher than those used during fermentation. Chaaban, H., Ioannou, I., Chebil, L., Slimane, M., Gerardin, C., Paris, C., Charbonnel, C., Chekir, L., and Ghoul, M. Effect of heat processing on thermal stability and antioxidant activity of six flavonoids. J. Food Process. Preserv. 41, E13203 (2017). This indicates that antioxidants can be added at any stage of fermentation including slurry, cook, liquefaction, propagation, and fermentation steps. In certain embodiments, the composition of the present invention is added during the fermentation step. According to at least one embodiment, the composition is added with the addition of yeast.

In certain embodiments, the composition includes at least one antioxidant, including but not limited to one or more plant-based antioxidant, in an amount ranging from about 1 mg/L to about 5000 mg/L, for instance from about 1 mg/L to about 1000 mg/L, such as about 1 mg/L to about 100 mg/L.

In certain embodiments, the composition includes at least one synthetic antioxidant in an amount ranging from about 0.0001 mg/L to about 100 mg/L, for instance from about 0.001 mg/L to about 100 mg/L, such as about 0.01 mg/L to about 10 mg/L.

In certain embodiments, the composition two or more antioxidants, such as one or more plant-based antioxidants, or in alternative embodiments at least one synthetic antioxidant, or blends thereof, where the additive is added to the fermentation process as a dry powder, liquid suspension, or solubilized in liquid including water or organic solvent.

In certain embodiments, the present invention is a method of increasing ethanol yield by at least 0.5%, for instance by at least 1%, at least 1.5%, or at least 2%, comprising adding a composition that contains an antioxidant in an amount effective to increase ethanol yield by at least 0.5%, for instance by at least 1%, at least 1.5%, or at least 2%. In certain embodiments, the composition is included in an amount sufficient to counteract heat or ethanol stress to yeast during the ethanol production process. In certain embodiments, the composition contains at least one plant-based antioxidant, for instance, at least one plant-based antioxidant selected from the group consisting of lutein, spearmint extract, green tea extract, acerola extract, rosemary extract, tocopherols, or blends thereof.

In certain embodiments, the present invention is a method of increasing ethanol yield by at least 0.5%, for instance by at least 1%, at least 1.5%, at least 2%. In certain embodiments, the composition is an additive that contains an antioxidant in an amount effective to increase ethanol yield by at least 0.5%, for instance by at least 1%, at least 1.5%, at least 2%. In certain embodiments, the additive is present in an amount effective to counteract heat or ethanol stress to yeast during the ethanol production process. For instance, in certain embodiments, the composition contains at least one synthetic antioxidant, such as TBHQ BHT, or blends thereof.

In certain embodiments, the present invention is a method of increasing ethanol yield by at least 0.5%, for instance by at least 1%, at least 1.5%, at least 2%, comprising adding a composition that contains a blend of antioxidants in an amount effective to counteract heat or ethanol stress to yeast during the ethanol production process. In certain embodiments, the composition contains one or more plant-based antioxidants. In alternative embodiments, the composition contains a plant-based antioxidant and a synthetic antioxidant. In certain embodiments, the composition is a blend of at least one plant-based antioxidant and at least one synthetic antioxidant.

In certain embodiments, the composition is considered “label-friendly” to consumers that may be sensitive to the inclusion of synthetic ingredients, particularly if the final product is for human consumption. For instance, in certain embodiments, the composition contains one or more plant-based antioxidants, or various blends thereof, and does not contain a synthetic antioxidant. In certain embodiments, the composition contains no synthetic antioxidant.

In certain embodiments, the addition of the composition does not impact the flavor profile of the final product, for instance, when the additive is added to the beer, wine, or other alcohol production process. According to certain embodiments, there was no impact on flavor profile of the final product when the composition was included in an amount ranging from about 1 to 5000 mg/L, for instance in an amount ranging from about 1 to 1000 mg/L or about 1 to 100 mg/L.

In certain embodiments, the present invention is a method of increasing ethanol yield comprising adding a composition that contains a blend of antioxidants in an amount effective to counteract heat or ethanol stress to yeast during the ethanol production process, wherein the composition is added during the fermentation step. In an alternative embodiment, the composition is added prior to the fermentation step.

In certain embodiments, the composition is added to a mixture of grain, water, and yeast in the fermentation vessel. In alternative embodiments, the composition is added to the grain and water mash.

In certain embodiments, the composition of the present invention is a dry powder that is added during the fermentation step. In alternative embodiments, the composition of the present invention is a liquid solution, or suspension.

In certain embodiments, the present invention is a method of mitigating heat and/or ethanol stress in yeast during ethanol production comprising adding a composition that contains an effective amount of an antioxidant to counteract heat and/or ethanol stress in yeast during fermentation. In certain embodiments, the composition contains at least one plant-based antioxidant. In certain embodiments, the at least one plant-based antioxidant is selected from the group consisting of lutein, spearmint extract, green tea extract, acerola extract, rosemary extract, tocopherols, or blends thereof.

In certain embodiments, the present invention is a method of mitigating heat and/or ethanol stress in yeast during ethanol production comprising adding a composition that contains an effective amount of an antioxidant to counteract heat and/or ethanol stress in yeast during fermentation. In certain embodiments, the composition contains at least one synthetic antioxidant. In certain embodiments, the at least one synthetic antioxidant is selected from the group consisting of TBHQ, BHT, or blends thereof.

In certain embodiments, the present invention is a method of increasing ethanol yield comprising adding a composition that contains an antioxidant in an amount effective to increase ethanol yield to a fermentation vessel that contains a mixture of grain, water, and yeast.

In certain embodiments, the present invention is a method of reducing overall costs in an industrial-scale ethanol production facility by adding a composition that contains an antioxidant in an amount effective to increase ethanol yield. In certain embodiments, the composition is added to a mixture of grain and water prior to the addition of yeast. In certain embodiments, the composition is added with the yeast. In certain embodiments, the composition is added to a fermentation vessel that contains a mixture of grain, water, and yeast.

### EXAMPLES

### Materials and Methods

Lutein (FloraGLO®): Lutein crystals were purified from marigolds and suspended in oil.
Acerola extract (Fortium® A): Acerola fruit grown from Malpighia emarginata, water extract.
Spearmint Extract (Neumentix®): Spearmint leaves grown from Mentha spicata L. (Kemin Industries, Inc. proprietary lines KI-MsEM0042, KI-MsEM0110), water extract.
Aqueous Tea Extract (AssuriTEA® Green): Tea leaves grown from Camellia sinensis, water extract.
Solvent Extracted Tea (Fortium® GT107): Tea leaves grown from Camellia sinensis and extracted with acetone and ethyl acetate, solvent extract.
Rosemary Extract (Fortium® M): Rosemary leaves grown from Rosmarinus officinalis extracted with either acetone and TFE or ethanol.
Mixed Tocopherols (Fortium® MT): Mixed tocopherols are extracted from vegetable oils with hexane, ethanol, and methanol.
Lipid-Soluble Green Tea (GT-FORT): Tea leaves grown from Camellia sinensis and extracted with ethyl acetate, dichloromethane, and ethanol.
Synthetic antioxidants: TBHQ (tert-butylhydroquinone) and BHT (butylated hydroxytoluene); tert-butlyhydroquinone was solubilized in propylene glycol and butylated hydroxytoluene was solubilized in ethanol. In alternative embodiments, the synthetic antioxidants are suspended in liquid or solubilized in liquid, including but not limited to water and organic solvents or combinations thereof.

### Example 1

Dry yeast was added to corn mash containing urea, protease, phytase, and glucoamylase. Lutein, a fat-soluble antioxidant, was added to the mash at 200, 1,000, or 2,000 mg/L. Dried aqueous tea extract or spearmint extract containing antioxidants were added to the corn mash at 100, 1,000, or 5,000 mg/L. Corn mash containing no added antioxidants was included in the experiment as a control group (Control). The corn mash was fermented in a temperature-controlled incubator shaker for 24 hours at 33° C. Following 24 hours, the yeast in the corn mash were subjected to heat stress by increasing the temperature to 38° C. and the corn mash fermented for another 24 hours for a total fermentation time of 48 hours. Ethanol levels were measured by HPLC.

Results: As summarized in FIG. 1, compared to Control with no added antioxidants, the addition of lutein increased ethanol content of corn mash by 4, 7, and 10% (200, 1,000, and 2,000 mg/L, respectively). As summarized in FIG. 2, corn mash with antioxidants from aqueous tea extract increased the ethanol content of the corn mash by 10, 11, and 6% (100, 1,000, and 5,000 mg/L, respectively) compared to Control. As summarized in FIG. 3, corn mash with added antioxidants from spearmint extract increased the ethanol content of the corn mash by 8, 13, and 3% (100, 1,000, and 5,000 mg/L, respectively) compared to Control.

### Example 2

Dry yeast was added to corn mash containing urea, protease, phytase, and glucoamylase. Tea or spearmint extracts containing antioxidants were added to the corn mash at 100 or 1,000 mg/L. Corn mash containing no added antioxidants was included in the experiment as a Control. The corn mash was fermented in a temperature-controlled incubator shaker for 24 hours at 33° C. Following 24 hours, the yeast in the corn mash were subjected to heat stress by increasing the temperature to 38° C. and the corn mash fermented another 48 hours for a total fermentation time of 72 hours. Ethanol levels were measured by HPLC.

Results: As summarized in FIG. 4, compared to Control with no added antioxidants, the addition of water-soluble antioxidants from aqueous tea extract or spearmint extract increased ethanol content of corn mash. Tea extract added to corn mash increased ethanol content by 16 and 12% (100 and 1,000 mg/L, respectively). As summarized in FIG. 5, corn mash with added water-soluble antioxidants from spearmint extract increased the ethanol content of the corn mash by 15 and 19% (100, and 1,000 mg/L, respectively) compared to Control.

### Example 3

Dry yeast was added to corn mash containing urea, protease, phytase, and glucoamylase. Solvent extracted tea (1.57 or 3.78 mg/L), spearmint extract (1.57 or 3.78 mg/L), or acerola extract (1.57 or 3.78 mg/L) containing water-soluble antioxidants were added to the corn mash. Corn mash containing no antioxidants was included in the experiment as a Control. The corn mash was fermented in a temperature-controlled incubator shaker for 24 hours at 33° C. Following 24 hours, the yeast in the corn mash were subjected to heat stress by increasing the temperature to 37° C. and the corn mash fermented for another 48 hours for a total fermentation time of 72 hours. Ethanol levels were measured by HPLC.

Results: As summarized in FIG. 6, compared to Control with no added antioxidants, the addition of water-soluble antioxidants from spearmint extract increased ethanol content of corn mash. Spearmint extract added to corn mash increased ethanol content by 3 and 0.2% (1.57 and 3.78 mg/L, respectively). As summarized in FIG. 7, compared to Control with no added antioxidants, the addition of water-soluble antioxidants from aqueous extracted acerola extracts increased ethanol content by 0.6% only at the 3.58 mg/L dose. As summarized in FIG. 8, compared to Control with no added antioxidants, the addition of solvent extracted antioxidants from tea to corn mash increased ethanol content in the corn mash by 2% in both the 1.57 and 3.78 mg/L groups.

### Example 4

Dry yeast was added to corn mash containing urea, protease, phytase, and glucoamylase. Rosemary extract (1, 10, or 100 mg/L), mixed tocopherols (1, 10, or 100 mg/L), and lipid soluble green tea (1, 10, or 100 mg/L) were added to the corn mash. Corn mash containing no antioxidants was included in the experiment as a Control. The corn mash was fermented in a temperature-controlled incubator shaker for 24 hours at 33° C. Following 24 hours, the yeast in the corn mash were subjected to heat stress by increasing the temperature to 37° C. and the corn mash fermented for another 48 hours for a total fermentation time of 72 hours. Ethanol levels were measured by HPLC.

Results: As summarized, in FIGS. 9-11, compared to Control with no added antioxidants, the addition of lipid-soluble antioxidants from rosemary, mixed tocopherols, and lipid soluble green tea increased ethanol content of corn mash over the first 24 hours of fermentation compared to control before heat stress was initiated. Rosemary extract increased ethanol content at 24 hour up to about 3%, mixed tocopherols up to about 4%, and lipid soluble green tea up to about 2%.

Following heat stress, the addition of lipid-soluble antioxidants from rosemary, mixed tocopherols, and lipid soluble green tea increased ethanol content of corn mash through 72 hours of fermentation compared to control. Rosemary extract increased ethanol content at 72 hour up to ˜2.6%, mixed tocopherols up to about 3.2%, and lipid soluble green tea up to about 3% (FIGS. 12-14).

### Example 5

Dry yeast was added to corn mash containing urea, protease, phytase, and glucoamylase. Solvent extracted tea (1, 10, or 100 mg/L) was added to the corn mash. Corn mash containing no antioxidants was included in the experiment as a Control. The corn mash was fermented in a temperature-controlled incubator shaker for 72 hours at 33° C. Ethanol levels were measured by HPLC.

Results: As summarized in FIG. 15, compared to Control with no added antioxidants, the addition of solvent extracted tea increased ethanol content of corn mash over 72 hours of fermentation compared to control at the 1 and 10 ppm doses (about 0.5 and 1.2%, respectively). The 100 mg/L dose was slightly lower than control (about 0.1%).

### Example 6

Dry yeast was added to corn mash containing urea, protease, phytase, and glucoamylase. tert-Butylhydroquinone (0.0001, 0.001, or 0.01 mg/L) and butylated hydroxytoluene (0.0001, 0.001, or 0.01 mg/L were added to the corn mash. Corn mash containing no antioxidants was included in the experiment as a Control. The corn mash was fermented in a temperature-controlled incubator shaker for 24 hours at 33° C. Following 24 hours, the yeast in the corn mash were subjected to heat stress by increasing the temperature to 37° C. and the corn mash fermented for another 48 hours for a total fermentation time of 72 hours. Ethanol levels were measured by HPLC.

Results: As summarized, in FIG. 16, compared to Control with no added antioxidants, the addition of the synthetic antioxidant tert-butylhydroquinone increased ethanol content of corn mash over the first 24 hours of fermentation compared to control at both the 0.0001 and 0.01 mg/L doses before heat stress was initiated. tert-Butylhydroquinone increased ethanol content at 24 hour up to about 0.4% (0.0001 mg/L) and up to about 1.8% (0.01 mg/L).

Following heat stress, the addition of tert-Butylhydroquinone increased ethanol content of corn mash through 72 hours of fermentation compared to control. tert-Butylhydroquinone increased ethanol content at 72 hour up to ˜2.8% (FIG. 16).

As summarized, in FIG. 17, compared to Control with no added antioxidants, the addition of the synthetic antioxidant tert-butylhydroquinone decreased ethanol content of corn mash over the first 24 hours of fermentation compared to control.

Following heat stress, the addition of butylated hydroxytoluene slightly decreased ethanol content of corn mash through 72 hours of fermentation compared to control. Butylated hydroxytoluene may not have been effective at increasing ethanol content due to steric hinderance around the hydroxyl group, lower resonance of the formed radical, as well as a less stable radical formed during oxidative stress under these conditions.

### Example 7

In order to assess whether or not the prototype impacts the flavor profile, beer was prepared using a home brewing kit (Brewer's Best, American Amber) following the recipe provided by the manufacturer. Water (2.5 gallons) was heated to 150-165° C. The grains (Cara Brown, 8 oz.) were added to the heated water and steeped for 20 minutes, then the heat was increased to bring the water mixture to a boil. Once at a boil, the malt ingredients (3.3 lb. amber liquid malt extract and 2.5 lb amber dry malt extract) and the first pack of hops (Willamette, 1 oz.) was added to the mixture. The mixture boiled for another 30 minutes, then a second pack of hops (Willamette, 1 oz.) was added, and the brew continued to boil for an additional 30 minutes. The heat was removed and the brew was allowed to cool to between 64-72° C. During the cooling step, an additional 2.5 gallons of room temperature water was added bringing the total volume to 5 gallons. The yeast (1 sachet) was added to the mixture and stirred to combine. The brew was divided into two glass containers to ferment. The first fermenter served as the negative control with no antioxidants added (“Control”), and 100 mg/L of green tea extract was added to the second fermenter (designated “Special”). Both fermenters were stored at room temperature. On Day 8, a blind taste test was performed and no difference in the flavor profile was detected between the two samples (Control and Special). The beer was then transitioned to glass bottles. A second blind taste test was performed on Day 12. No difference in flavor profile was detected. A third blind taste test was performed on Day 20, and again, no difference in flavor profile was detected.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

It should be further appreciated that minor dosage and formulation modifications of the composition and the ranges expressed herein may be made and still come within the scope and spirit of the present invention.

It is also to be understood that the formulations and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise. Where a range of values is provided, it is understood that each intervening value, to the tenth of the unit of the lower limit unless the context clearly dictates otherwise, between the upper and lower limit of that range, and any other stated or intervening value in that stated range, is encompassed within the scope of the present disclosure. The upper and lower limits of these smaller ranges may independently be included in the smaller ranges, and are also encompassed within the scope of the present disclosure, subject to any specifically excluded limit in the stated range. Where the stated range includes one or both of the limits, ranges excluding either or both of those included limits are also included in the scope of the present disclosure. All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, 10) contained within the range. In this specification and the appended claims, the singular forms “a,” “an” and “the” include plural reference unless the context clearly dictates otherwise. All combinations of method steps or process steps as used herein can be performed in any order, unless otherwise specified or clearly implied to the contrary by the context in which the referenced combination is made.

To the extent that the terms “contains” or “includes” or “including” or “have” or “having” are used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A” or “B” or both “A” and “B”. When the Applicant intends to indicate “only A or B but not both” then the term “only A or B but not both” or similar structure will be employed. Thus, use of the term “or” herein is the inclusive, and not the exclusive use. Also, to the extent that the terms “in” or “into” are used in the specification or the claims, it is intended to additionally mean “on” or “onto.” In this specification and the appended claims, the singular forms “a,” “an” and “the” include plural reference unless the context clearly dictates otherwise.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives. It should be appreciated that in some exemplary embodiments, well-known processes, well-known methods, devices, and technologies are not described in detail. Persons of ordinary skill in the art will understand that modifications and variations of the embodiments disclosed can be made within the scope of the present invention in order to achieve substantially similar results.

### REFERENCES INCORPORATED HEREIN IN THEIR ENTIRETY

1. Jacques, K., Lyons, T., and Kelsall, D. 2003, The Alcohol Textbook, 4^{th} Edition, Nottingham University Press, United Kingdom.
2. Birch, R. M., and Walker, G. M. (2000) Influence of magnesium ions on heat shock and ethanol stress responses of Saccharomyces cerevisiae. Enzyme Microb Technol 26, 678-687
3. Geddes, C. (2017) Running hot: 7 tips for warm weather ethanol fermentation. In Biofuels Digest
4. Stanley, D., Bandara, A., Fraser, S., Chambers, P. J., and Stanley, G. A. (2010) The ethanol stress response and ethanol tolerance of Saccharomyces cerevisiae. J Appl Microbiol 109, 13-24
5. Earls, D. (2017) How EtOH production affects yeast health: 4 tips for dealing with ethanol stress. In Biofuels Digest
6. Sanchez, Y., Taulien, J., Borkovich, K. A., and Lindquist, S. (1992) Hsp104 is required for tolerance to many forms of stress. EMBO J 11, 2357-2364
7. Chaaban, H., Ioannou, I., Chebil, L., Slimane, M., Gerardin, C., Paris, C., Charbonnel, C., Chekir, L., and Ghoul, M. Effect of heat processing on thermal stability and antioxidant activity of six flavonoids. J. Food Process. Preserv. 41, E13203 (2017)
8. Piper, P. W. (1993) Molecular events associated with acquisition of heat tolerance by the yeast Saccharomyces cerevisiae. FEMS Microbiol Rev 11, 339-355
9. Dani, C., Bonatto, D., Salvador, M., Pereira, M. D., Henriques, J. A., and Eleutherio, E. (2008) Antioxidant protection of resveratrol and catechin in Saccharomyces cerevisiae. J Agric Food Chem 56, 4268-4272
10. Caridi, A. (2003) Effect of protectants on the fermentation performance of wine yeasts subjected to osmotic stress. Food Technology and Biotechnology 41, 145-148
11. Chong, S. Y., Chiang, H. Y., Chen, T. H., Liang, Y. J., and Lo, Y. C. (2019) Green tea extract promotes DNA repair in a yeast model. Sci Rep 9, 3842
12. Morano, K. A., Grant, C. M., and Moye-Rowley, W. S. (2012) The response to heat shock and oxidative stress in Saccharomyces cerevisiae. Genetics 190, 1157-1195
13. Lebaka, V., Ryu, H., and Wee, Y. (2014) Effect of fruit pulp supplementation on rapid ethanol production in very high gravity (VHG) fermentation. Bioresources and Bioprocessing 1
14. Li, H., Wang, L., and Luo, Y. (2018) Composition analysis by UPLC-PDA-ESI (−)-HRMS and antioxidant activity using Saccharomyces cerevisiae model of herbal teas and green teas from Hainan. Molecules 23
15. Maeta, K., Nomura, W., Takatsume, Y., Izawa, S., and Inoue, Y. (2007) Green tea polyphenols function as prooxidants to activate oxidative-stress-responsive transcription factors in yeasts. Appl Environ Microbiol 73, 572-580
16. Mendes, V., Vilaca, R., de Freitas, V., Ferreira, P. M., Mateus, N., and Costa, V. (2015) Effect of myricetin, pyrogallol, and phloroglucinol on yeast resistance to oxidative stress. Oxid Med Cell Longev 2015, 782504
17. Naparlo, K., Zyracka, E., Bartosz, G., and Sadowska-Bartosz, I. (2018) P-277—Influence of catechins on Saccharomyces cerevisiae subjected to thermal stress. Free Radical Biology and Medicine 120, S129
18. Pan, S., Jia, B., Liu, H., Wang, Z., Chai, M. Z., Ding, M. Z., Zhou, X., Li, X., Li, C., Li, B. Z., and Yuan, Y. J. (2018) Endogenous lycopene improves ethanol production under acetic acid stress in Saccharomyces cerevisiae. Biotechnol Biofuels 11, 107
19. Subramaniyan, S., and Dyavaiah, M. (2018) Evaluation of antioxidant activity of magnolol in Saccharomyces cerevisiae. International Journal of Pharmacy and Pharmaceutical Sciences 10, 104-107
20. Burphan, T., Tatip, S., Limcharoensuk, T., Kangboonruang, K., Boonchird, C., and Auesukaree, C. (2018) Enhancement of ethanol production in very high gravity fermentation by reducing fermentation-induced oxidative stress in Saccharomyces cerevisiae. Sci Rep 8, 13069
21. Prakash, A., and Baskaran, R. (2018) Acerola, an untapped functional superfruit: a review on latest frontiers. J Food Sci Technol 55, 3373-3384

1. A method of increasing ethanol yield comprising adding a composition that contains an antioxidant in an amount effective to increase ethanol yield by at least 0.5% during an ethanol production process.

2. The method of claim 1, wherein the composition contains at least one plant-based antioxidant.

3. The method of claim 2, wherein the at least one plant-based antioxidant is selected from the group consisting of lutein, spearmint extract, green tea extract, rosemary extract, acerola extract, tocopherols, or blends thereof.

4. The method of claim 1, wherein the composition contains at least one synthetic antioxidant.

5. The method of claim 4, wherein the at least one synthetic antioxidant is selected from the group consisting of TBHQ, BHT, or blends thereof.

6. The method of claim 1, wherein the composition is added during the fermentation step.

7. The method of claim 1, wherein the composition is added prior to the fermentation step.

8. The method of claim 1, wherein the composition is added to a mixture of grain, water, and yeast.

9. The method of claim 1, wherein the composition is a dry powder, liquid solution, or suspension.

10. The method of claim 1, wherein the composition is a blend of at least one plant-based antioxidant and at least one synthetic antioxidant.

11. A method of mitigating heat and/or ethanol stress in yeast during ethanol production comprising adding a composition that contains an effective amount of an antioxidant to counteract heat and/or ethanol stress in yeast during fermentation.

12. The method of claim 11, wherein the composition contains at least one plant-based antioxidant.

13. The method of claim 12, wherein the at least one plant-based antioxidant is selected from the group consisting of lutein, spearmint extract, green tea extract, rosemary extract, acerola extract, tocopherols, or blends thereof.

14. The method of claim 11, wherein the composition contains at least one synthetic antioxidant.

15. The method of claim 14, wherein the at least one synthetic antioxidant is selected from the group consisting of TBHQ, BHT, or blends thereof.

16. The method of claim 11, wherein the composition is added during the fermentation step.

17. The method of claim 11, wherein the composition is added prior to the fermentation step.

18. The method of claim 11, wherein the composition is a dry powder, liquid solution, or suspension.

19. The method of claim 11, wherein the composition is a blend of at least one plant-based antioxidant and at least one synthetic antioxidant.

20. A method of increasing ethanol yield comprising adding a composition that contains an antioxidant in an amount effective to increase ethanol yield to a fermentation vessel that contains a mixture of grain, water, and yeast.

21. An additive for increasing ethanol yield comprising a composition that contains two or more antioxidants in an amount effective to increase ethanol yield by at least 0.5%.

22. The additive of claim 21 wherein the two or more antioxidants are selected from the group consisting of lutein, spearmint extract, green tea extract, rosemary extract, acerola extract, tocopherols, TBHQ BHT, or blends thereof.
